(19) United States
(12) Patent Application Publication
(10) Pub. No.: US 2024/0159754 A1
(43) Pub. Date: May 16, 2024

(54) LUNG CANCER DIAGNOSIS USING BIOMARKERS FROM EXOSOMES

(71) Applicant: TERASOM S.R.O., Praha (CZ)

(72) Inventor: Daniela PANKOVA, Prague (CZ)

(21) Appl. No.: 18/280,866

(22) PCT Filed: Mar. 7, 2022
(86) PCT No.: PCT/EP2022/055698

§ 371 Date: Sep. 7, 2023

(30) Foreign Application Priority Data
Mar. 8, 2021 (GB) 2103200.8

(51) Int. Cl. G01N 33/574; G01N 33/543

(52) U.S. Cl. CPC G01N 33/57423; G01N 33/54387

(57) ABSTRACT

The present invention relates to the diagnosis of lung cancer in a subject. The invention provides a diagnostic method of obtaining an indication of the presence of lung cancer in a subject, based on the presence of one or more biomarkers which are present in or on the surface of exosomes or are cleaved exosomal surface polypeptides obtained from a liquid biopsy or other biological sample. The invention also provides devices, such as flow and microfluidics devices, which may be used for diagnosis of lung cancer in a subject.

The present invention relates to the diagnosis of lung cancer in a subject. The invention provides a diagnostic method of obtaining an indication of the presence of lung cancer in a subject, based on the presence of one or more biomarkers which are present in or on the surface of exosomes or are cleaved exosomal surface polypeptides obtained from a liquid biopsy or other biological sample. The invention also provides devices, such as flow and microfluidics devices, which may be used for diagnosis of lung cancer in a subject.

Lung cancer is the most frequently diagnosed cancer (Fan et al., 2015) and it remains the leading cause of cancer-related death worldwide in men and women (Siegel et al., 2017). Despite of development of new therapeutic methods, the general prognosis of lung cancer patients is poor, partly because the majority of cases are detected in the late stages when metastases have spread into the lymph nodes and other parts of the body. These advanced stages of lung cancer are difficult to surgically resect and are associated with an increased rate of post-operative lung cancer recurrence (Kumarakulasinghe et al., 2015). Five-year survival of patients with advanced lung cancer is only around 4% (National Lung Screening Trial Research Team et al., 2011), whilst the 5-year survival rate is 54% in patients diagnosed in early stages that have a primary tumour which is still operable and localized into the lung.

Standard diagnostic procedures for the detection of lung cancer include chest X-ray, CT (computerized tomography) scan and tissue biopsy. These are unspecific and have numerous limitations that confine the assessment of disease to the early stages. The non-invasive X-ray method is only able to detect tumours larger than 1 cm; therefore, it can take several years for a lung tumour to reach the size at which it can be identified. CT scans provide more information and can detect smaller tumours than X-ray. However, the waiting period is usually longer and during this time aggressive lung tumours can often double their size or start to metastasize. Moreover, tumours suspected by these medical imaging techniques require further investigation to confirm a diagnosis of lung cancer.

Lung cancer biopsies are also often inaccurate due to tumour heterogeneity (Levy et al., 2016). Furthermore, the accessibility of the tumour biopsy represents an additional problem as more than 80% of lung cancer patients with advanced stages have only limited or no tissue available from small biopsies or cytology to perform further investigations; moreover, obtaining tissue biopsy from the patient is very invasive, time consuming and error prone (Wong et al., 2014).

Liquid biopsy is an alternative method to tissue biopsy. Liquid biopsy recently became accepted as a new tool for cancer detection. This technique is not only minimally invasive for patients, but also provides a valuable source of fresh tumour-derived material from the bloodstream that better reflects genetic and molecular information on the primary tumour and any metastatic sites. Generally, a sample from a liquid biopsy requires only 10 ml of blood or other body fluids; this enables the capture of circulating tumour cells (CTCs), cell-free RNA (cfRNA), cell-free DNA (cfDNA), circulating tumour DNA (ctDNA) and exosomes.

The highest accuracy of commercially-available tests allow the detection of ctDNA in 72% of lung cancer patients in stage II-IV (Cristiano et al., 2019) with a strong correlation between ctDNA level and tumour volume (Newman et al., 2014). However, although the detection of these cells or nucleic acids fragments offers a promising tool for the diagnosis of lung cancer, their processing requires long procedures including ultra-centrifugation, extraction, analyses of sequences and sensitive methods for the separation of ctDNA from normal cells cfDNA. Moreover, tumour ctDNA is not stable and has only 2 hours of half-life; this means that samples must be evaluated quickly and this can cause false negative results in the lung cancer patient (Diehl et al., 2008).

The recent growing interest in exosomes isolated from liquid biopsy arises from their potential clinical application as biomarkers for cancer detection and as a therapeutic drug-delivery system. Exosomes are cellular membrane-derived extracellular vesicles between 30-150 nm in size, with lipid bilayer membranes, which are secreted by various eukaryotic cells. Their existence was first confirmed by Turbide's group in the late 1980s and they were initially considered as cellular waste with no significant biological role (Johnstone et al., 1987).

Over the past two decades, however, exosomes have been extensively investigated for their unique role in intercellular communication and many other cellular processes. Exosomal biological functions depend on their biologically active cargos; these differ greatly between the parental cells from which they are secreted. According to one exosome database, exosomal cargoes contain up to 9769 different proteins, 3408 different mRNAs, 2838 different miRNAs and 1116 different lipids (www.exocarta.orq); this makes exosomes potential biomarkers for cancer diagnosis.

The cancer-derived exosomes are secreted by a tumour's tissue and can be isolated from various body fluids, including blood, saliva or urine (van der Pol et al., 2012); they therefore provide important information about a tumour's biological profile, metastatic capacity or growth rate. The lipid bilayer membrane protects exosomes from extreme pH and degradation by ribonucleases during their circulation in the bloodstream, and provides exosomes with a longer lifespan and higher stability compared to cell-free RNA (Sourvinou et al., 2013).

However, although different isolation methods have been described, the isolation of exosomes from clinical samples can be very challenging. Standard techniques include a combination of centrifugation and ultracentrifugation, density gradient separation, chromatography or immunoprecipitation based on specific antibodies coated beads (Vanni et al., 2017). These methods are usually time consuming; moreover, physical isolation can alter the structure of the exosomes.

The targeting of tumour exosomal biomarkers from liquid biopsies must be highly specific to enable their clinical applications. The biggest challenge is to detect even small pathological changes from exosomes with minimal sample preparation and cost. Over the past few years, exosomal biomarkers from liquid biopsies (e.g. blood, saliva, urine) have been investigated for use in lung cancer diagnosis and screening.

Evaluation of exosomal microRNAs (Cazzoli et al., 2013), RNA, long non-coding RNA MALAT-1 (Zhang et al., 2017), miR-184 (Song et al., 2018), lipids (Fan et al., 2018) and exosomal proteins (Jakobsen et al., 2015) have been proposed for early and advanced stages of lung cancer. Furthermore, the exosomal membrane surface proteins CD91, CD317 and EGFR have been suggested as potential tumour markers (Yamashita et al., 2013); however, they have not shown precise specificity for lung cancer detection. Recent LC-MS/MS analyses of exosomes from the saliva of lung cancer patients initially identified four potential candidates, namely BPIFA1, CRNN, MUC5B and IQGAP proteins for potential detection of lung cancer; however, further analyses showed no significant differences in saliva between lung cancer and control patients (Sun et al., 2018).

Proteomic nano-HPLC-chip-MS/MS profiling from urine in non-small lung cancer patients revealed higher expression levels of LRG1 protein and suggested another potential candidate for diagnosis of NSCLC (Li et al., 2011).

Targeting specific surface exosomal tumour biomarkers from liquid biopsies may provide a high diagnostic potential with minimal sample preparation in lung oncology. However, little is currently known about the biogenesis of exosomes, particularly about how proteins from parental cells are processed inside or on the surface of exosomes. For example, cell surface proteins could be processed into the exosomes and yet not be expressed on the exosome surface; on the other hand, cell cytoplasmic proteins could be inserted into exosomal membranes where they may act as surface exosomal biomarkers. A further issue is that some exosomal surface proteins are expressed in many different types of cancer exosomes, not only in lung cancer.

For these reasons and others, the detection of lung cancer by specific surface exosomal biomarkers from liquid biopsies has not yet been translated in routine clinical practice; and there are currently no available exosomal biomarkers which can accurately distinguish a certain type of the cancer.

There is a need therefore for exosomal membrane surface proteins to be identified which can be used to distinguish between different types of cancer and/or to detect early stages of lung cancer in a patient. A group of proteins has now been identified as being specific to the surface of exosomes from lung cancers. These biomarkers may be used for the detection of lung cancer in liquid samples from lung cancer patients. Furthermore, antibodies against these biomarkers or combinations thereof may be used in diagnostic devices for the detection of lung cancer.

It is an object of the invention to provide a method of obtaining an indication of the presence of lung cancer and/or metastatic stage in a subject. It is another object of the invention to provide a device, e.g. a lateral flow device, vertical flow device or microfluidics devices, which can be used to provide an indication of the presence of lung cancer biomarkers in a biological sample.

In one embodiment, the invention provides a method of obtaining an indication of the presence of lung cancer in a subject, the method comprising the step:
(a) determining the presence of one or more biomarkers selected from the group consisting of PLD3, MAGE4A, GAGE2D, MTAP and UCHL1 polypeptides within a biological sample obtained from the subject, wherein the biological sample comprises exosomes and/or polypeptides obtained therefrom,

wherein the presence of one or more of the biomarkers within the biological sample is indicative of the presence of lung cancer in the subject.

In another embodiment, the invention provides a method of distinguishing between early stage lung cancer (e.g. stages I, II, III) and metastatic lung disease in a subject or of determining the stage of the lung cancer in the subject, the method comprising the step:
(a) determining the presence of or level of one or more biomarkers selected from the group consisting of PLD3, MAGE4A, GAGE2D, MTAP and UCHL1 polypeptides within a biological sample obtained from the subject, wherein the biological sample comprises exosomes and/or polypeptides obtained therefrom,

wherein

the presence of PLD3, MTAP and/or UCHL1 within the biological sample is indicative of

the presence of an early stage lung cancer in the subject, and

the presence of MAGE4A and/or GAGE2D in the biological sample is indicative of the presence of metastatic lung disease in the subject.

In general, the methods of the invention are carried out in vitro or ex vivo (unless the context requires otherwise, e.g. wherein the method includes administration steps).

Lung cancer, also known as lung carcinoma, is a malignant lung tumour characterized by uncontrolled cell growth in tissues of the lung. This growth can spread beyond the lungs by the process of metastasis into nearby tissue or other parts of the body. Most cancers that start in the lungs, known as primary lung cancers, are carcinomas.

In some embodiments, the lung cancer is small-cell lung carcinoma (SCLC). In other embodiments, the lung cancer is non-small-cell lung carcinoma (NSCLC). The three main subtypes of NSCLC are adenocarcinoma, squamous-cell carcinoma, and large-cell carcinoma. Rare subtypes include pulmonary enteric adenocarcinoma.

Preferably, the NSCLC is adenocarcinoma (NSLC).

The methods of the invention may also be used to obtain an indication of the presence of a metastasis derived from a lung cancer in the subject. The lung cancer may be stage I, 1A, 1B, II, IIA, IIB, III or IV.

The early stage lung cancer may be a non-metastatic stage.

As used herein, the term “is indicative of the subject having lung cancer” means that there is a positive correlation between the presence or different (e.g. increased) levels of one or more biomarkers and the presence of lung cancer in that subject.

Consequently, the presence of or different/increased levels of one or more biomarkers in exosomes from the subject means an increased likelihood or statistically-significant chance of the subject having lung cancer. Significance may be measured by any suitable technique, e.g. Student's t-test (p<0.05).

The subject is preferably a human subject. The subject may be male or female.

The subject may be alive or dead (i.e. the method may be used for post-mortem diagnosis). The human may, for example, be 0-10, 10-20, 20-30, 30-40, 40-50, 50-60, 60-70, 70-80, 80-90, 90-100 or above 100 years old. The human may be one who is at risk from a particular disease or disorder, e.g. lung cancer, or one who has previously suffered from a particular disease or disorder, e.g. lung cancer.

A control subject may be defined as a non-diseased subject, a subject without lung cancer, or a healthy-aged subject.

Preferably, the biological sample is a bodily fluid or a liquid biopsy from the subject. Preferably, the biological sample is a sample of the subject's blood, saliva, bronchial lavage or urine. More preferably, the biological sample is a sample of the subject's blood. Most preferably, the biological sample is blood serum or blood plasma.

In some embodiments, the method additionally comprises the step, prior to Step (a), of obtaining one or more biological samples from the subject. In some embodiments, one or more of the biomarkers are detected directly in the biological sample, e.g. from within a sample of the subject's blood, blood serum or blood plasma.

In other embodiments, exosomes are first isolated and/or purified from the biological sample before the biomarkers are detected. The biological sample may therefore comprise isolated and/or purified exosomes.

Exosomes may be isolated and/or purified from the biological samples by any suitable method. Such methods include centrifugation or ultracentrifugation; this may or may not be combined with size exclusion chromatography. Size exclusion chromatography columns may be used, for example using porous gel columns. In such columns, the pore size is preferably 30-70 nm in order to allow the passage of exosomes but not larger vesicles. Other methods include immunoprecipitation by using exosomal markers (e.g. CD9/CD63). This may be achieved, for example, by direct immunoprecipitation by commercially available kits.

In other embodiments, the exosomes are isolated and/or purified within a vertical flow device, a lateral flow device or a microfluidics device. In some embodiments, one or more of the biomarkers are detected directly in the isolated and/or purified exosomes.

In other embodiments, exosomal polypeptides (preferably exosomal surface polypeptides) are first isolated and/or purified from the isolated and/or purified exosomes.

Polypeptides may be isolated and/or purified from the isolated and/or purified exosomes by any suitable method. Preferably, membrane associated polypeptides are isolated and/or purified from the isolated and/or purified exosomes. Membrane associated polypeptides may be isolated by the use of a suitable detergent, e.g. sodium deoxycholate.

In some embodiments, the exosomes are treated to release polypeptides from the outer surfaces of the exosomes. As used herein, the term “released” means that whole or parts of polypeptides which are present on the outer surface of the exosomes become no longer bound to the exosome surface and are thus able to move independently of the exosomes.

The exosomes may be treated with a protease. Preferably, the protease is a serine protease, more preferably, the protease is trypsin. Trypsin cuts polypeptide chains mainly at the carboxyl side of the amino acids lysine or arginine. A concentration of trypsin is used which releases some or the majority of surface-anchored polypeptides from the exosomes without causing undue degradation of those polypeptides. Suitable concentrations include 0.25% or 0.5%, for example, for 30 minutes at 37′C.

Polypeptides which are isolated and/or purified from exosomes and/or polypeptides which have been released from the outer surfaces of the exosomes may subsequently be purified and/or concentrated by any suitable method, e.g. by precipitation. Examples of suitable precipitation methods include using trichloroacetic acid (TCA) or ammonium sulphate. The polypeptides and/or surface polypeptides may also be immuno-precipitated (e.g. with biomarker-specific antibodies). Preferably, the polypeptides and/or surface polypeptides are precipitated using TCA. It is particularly preferred that surface polypeptides are concentrated prior to determining the presence of UCHL1 or PLD3.

The presence of one or more of biomarkers selected from the group consisting of PLD3, MAGE4A, GAGE2D, MTAP and UCHL1 is detected within the biological sample. As used herein, the term “biological sample” includes exosomes which have been isolated and/or purified; and polypeptides and surface polypeptides which have been isolated from the isolated and/or purified exosomes.

The MAGE1 gene encodes Melanoma-associated antigen 4. The human MAGE1 gene has the UniProtKB database accession no. P43358 (MAGA4_HUMAN).

The GAGE2D gene encodes G antigen 2D. The human GAGE2D gene has the UniProtKB database accession no. Q9UEU5 (GGE2D_HUMAN).

The MTAP gene encodes S-methyl-5′-thioadenosine phosphorylase. The human MTAP gene has the UniProtKB database accession no. Q13126 (MTAP_HUMAN).

The PLD3 gene encodes a 5′-3′ exonuclease. The human PLD3 gene has the UniProtKB database accession no. Q81V08 (PLD3_HUMAN).

The UCHL1 gene encodes Ubiquitin carboxyl-terminal hydrolase isozyme L1. The human UCHL1 gene has the UniProtKB database accession no. P09936 (UCHL1_HUMAN).

TPGB is broadly expressed on the surface of exosomes in various cancer cell types; it may therefore be used as a positive control. The method of the invention may therefore additionally comprise the step of determining the presence of the TPGB polypeptide in the biological sample. The TPGB gene encodes Trophoblast glycoprotein. The human TPGB gene has the UniProtKB database accession no. Q13641 (TPBG_HUMAN). It is also known as 5T4 oncofetal antigen.

One method of the invention includes the step of determining the presence or elevated level or concentration of one or more of the specified biomarkers within the biological sample. In some embodiments, the presence of 1, 2, 3, 4, or 5 of the biomarkers may be determined. Preferably, the presence of 2-3 of the biomarkers is determined.

For example, the presence of the following combinations of biomarkers may be determined using any combination of the 5 biomarkers, including:
MAGE4A, GAGE2D, MTAP, PLD3, UCHL1;
MAGE4A, GAGE2D, MTAP, PLD3,
MAGE4A, GAGE2D, MTAP, UCHL1;
MAGE4A, GAGE2D, PLD3, UCHL1;
MAGE4A, MTAP, PLD3, UCHL1;
GAGE2D, MTAP, PLD3, UCHL1;
MAGE4A, GAGE2D, MTAP,
MAGE4A, GAGE2D, PLD3,
MAGE4A, GAGE2D, UCHL1;
MAGE4A, MTAP, PLD3,
MAGE4A, MTAP, UCHL1;
MAGE4A, PLD3, UCHL1;
GAGE2D, MTAP, PLD3,
GAGE2D, MTAP, UCHL1;
MTAP, PLD3, UCHL1;
MAGE4A, GAGE2D,
MAGE4A, MTAP,
MAGE4A, PLD3,
MAGE4A, UCHL1;
GAGE2D, MTAP,
GAGE2D, PLD3,
GAGE2D, UCHL1;
MTAP, PLD3,
MTAP, UCHL1; and
PLD3, UCHL1;
PLD3;
UCHL1;
MTAP;
MAGE4A;
GAGE2D;

In some preferred embodiments, the biomarkers are (i) PLD3, (ii) MTAP, or (iii) PLD3 and MTAP.

TPGB (5T4) may be added to any of the above combinations (as a positive control). CD81, CD9 or other exosomal markers may also be used as markers for isolated exosomes via microfluidics, lateral or vertical flow devices.

The presence or levels of the biomarkers may be determined by any suitable means, preferably by immuno-detection using labelled biomarker-specific antibodies, e.g. by Western blot, ELISA or a lateral flow device.

Antibodies against all of the specified biomarkers are commercially available, e.g.
MAGE4A: cat. no: 12508-1-AP; Proteintech, USA;
GAGE2D; cat. no: 12532-1-AP, Proteintech, USA;
MTAP, cat. no: PA5-22000, Invitrogen, USA;
PLD3, cat. No: PA5-104016, Invitrogen, USA;
UCHL1, cat. no: CF504289, Origene, USA; and
5T4, cat. no: ab-129058, Abcam, USA.

In another embodiment, the invention provides a method of obtaining an indication of the prognosis of lung cancer in a subject, the method comprising the steps:
a) determining the presence or level of one or more biomarkers within a biological sample obtained from the subject at a first time point; and
b) determining the presence or level of one or more of the same biomarkers within a biological sample (of the same type) obtained from the subject at a second (later) time point;

wherein the biomarkers are selected from the group consisting of PLD3, MAGE4A, GAGE2D, MTAP, and UCHL1 polypeptides,

wherein the biological samples both comprise exosomes and/or polypeptides obtained therefrom,

wherein an increase in the level of one or more of the biomarkers within the second biological sample compared to the corresponding level(s) of biomarkers in the first biological sample is indicative of a negative prognosis of the subject, and

wherein a decrease in the level of one or more of the biomarkers within the second biological sample compared to the corresponding level(s) of biomarker(s) in the first biological sample is indicative of an improvement in the prognosis of the subject.

The second time point is after the first time point. The first time point may, for example, be at an early stage in the lung cancer (e.g. stages IA, IB, IIA or IIB). The second time point may be at a later stage in the lung cancer (stage III or stage IV); or after the subject has been treated with medicament suitable for the treatment of lung cancer. The first and second time points may be any suitable time intervals, e.g. at least one week apart, 1-12 months apart, or at least 1, 2, 3, 4 or 5 years apart.

The samples of exosomes and/or polypeptides obtained in Steps (a) and (b) must be directly comparable, i.e. the same biomarkers are compared and the biological samples must both be of the same type (e.g. both are blood samples) and treated in the same manner.

It is recognised that lung cancer is not merely one disease but a general term for a number of associated disorders. The invention may therefore be used to stratify subjects into such associated disorders or lung cancer subgroups or to identify the lung cancer stage.

In a further embodiment, therefore, the invention provides a method of classifying a subject into a lung cancer subgroup, the method comprising the steps:
(a) determining the presence or level of one or more biomarkers selected from the group consisting of PLD3, MAGE4A, GAGE2D, MTAP and UCHL1 polypeptides within a biological sample obtained from the subject, wherein the biological sample comprises exosomes and/or polypeptides obtained therefrom; and
(b) classifying the subject as belonging to a lung cancer subgroup based on the presence of or level of one or more of the biomarkers within the biological sample.

The classification in Step (b) may be made using corresponding biomarker presences or levels from other subjects who have previously been identified as belonging to a specific subgroup. For example, the lung cancer subgroup may be a lung cancer stage as referred to hereinabove. The classification of the subjects may also be used to select subjects for clinical trials.

The presence or levels of one or more of the biomarkers within a biological sample may be used to quantify the severity of a lung cancer in that subject. This value may therefore be used to determine whether or not a particular drug is having a beneficial effect on the treatment of the subject.

In a further embodiment, therefore, the invention provides a method of obtaining an indication of the efficacy of a drug which is being used to treat lung cancer in a subject, the method comprising the steps:
a) determining the presence or level of one or more biomarkers within a biological sample obtained from the subject at a first time point;
b) determining the presence or level of one or more of the same biomarkers within a biological sample (of the same type) obtained from the subject at a second (later) time point,

wherein the biomarkers are selected from the group consisting of PLD3, MAGE4A, GAGE2D, MTAP and UCHL1 polypeptides,

wherein the biological samples both comprise exosomes and/or polypeptides obtained therefrom,

wherein a drug has been administered to the subject in the interval between the first and second time points,

wherein an increase in the level of one or more of the biomarkers within the second biological sample compared to the corresponding level(s) of biomarker(s) in the first biological sample is indicative of a lack of efficacy of the drug, and

wherein a decrease in the level of one or more of the biomarkers within the second biological sample compared to the corresponding level(s) of biomarker(s) in the first biological sample is indicative of the efficacy of the drug.

The biological samples obtained in Steps (a) and (b) must be directly comparable, i.e. the biological samples must both be of the same type (e.g. both are blood samples) and subsequently treated in the same manner. The first time point may, for example, be at an early stage in the lung cancer (e.g. stages IA, IB, IIA or IIB). The second time point may be at a later stage in the lung cancer (stage III or stage IV).

In another embodiment, the invention provides a method of treating lung cancer in a subject, the method comprising the steps of:
(a) obtaining an indication of the presence of lung cancer in a subject by a method of the invention; and
(b) administering a treatment appropriate for treating lung cancer to the subject if an indication of the presence of lung cancer in the subject is obtained, thereby treating lung cancer in the subject.

In another embodiment, the invention provides a method of treating lung cancer in a subject, the method comprising the step of:
(a) administering a treatment appropriate for treating lung cancer to the subject, wherein, prior to administration, an indication of the presence of lung cancer in the subject, has been obtained by a method of the invention.

Lateral flow devices (LFDs) are often used to test a liquid sample, such as saliva, blood or urine, for the presence of an analyte. Examples of lateral flow devices include home pregnancy tests, home ovulation tests, tests for other hormones, tests for specific pathogens and tests for specific drugs. For example, EP 0291194 describes a lateral flow device for performing a pregnancy test.

The features of lateral flow devices are well known in the art. Reference may be made, for example, to the following which describe general features of lateral flow devices, including methods of their production, and methods of linking detectable labels and immobilising reagents: EP2453242, US2015176050, WO 2020/049444, US 2020/0023354 A1, JP 2019023647 A, EP 0291194 A1, WO 2020/033235 A1, WO2019122816 (A1), WO 2019/023597, US 2020132693 A1, WO 2020/041267 A2, US 2018/372733 (A1), US 2018/133343 (A1), US2016017065 (A1), the contents of which are all specifically incorporated herein by reference.

Lateral flow devices generally include one or more of the following discrete zones (a)-(c), and optionally (d) and (e), which are in fluid communication with one another, optionally in this order.

(a) A sample receiving zone. This zone receives the test sample comprising the analyte (biomarker) to be tested for.

The sample receiving zone may comprise or may precede a size exclusion zone (i.e. the size exclusion zone may precede or be part of the sample receiving zone). In some embodiments, the size exclusion zone may precede or be part of the detection zone.

The size exclusion zone comprises a zone wherein exosomes from the biological sample are isolated from the other components (e.g. cells) of the sample based on exosome size. The size exclusion zone allows the passage of exosomes based on exosome size.

For example, the size exclusion zone may be one which only allows the passage of exosomes whose largest dimension is 30-150 nm, e.g. 30-70, 70-100 or 100-150 nm. The size exclusion zone may, for example, comprise a porous-gel material having a pore size of 30-150 nm, e.g. 30-70, 70-100 or 100-150 nm.

Examples of suitable porous-gel materials include polysaccharide resin, sucrose, dextran, silica-based porous material, polyacrylamide gel, agarose, cellulose or combinations thereof. Preferably, these materials have pore size of 30-150 nm.

In some embodiments, the sample is applied directly onto the detection zone and a transfer fluid (without a sample) may be applied to the sample receiving zone. In such embodiments, the detection zone may comprise or be preceded by a size exclusion zone, as defined above.

(b) A conjugate zone. This zone comprises one or more first specific binding partners for the analytes (biomarkers). Each first specific binding partner is linked to a detectable label. The first specific binding partners are not immobilised in the conjugate zone; they are capable of being mobilised, i.e. being transported to subsequent zones by capillary action or active fluid flow.

The labelled first specific binding partners are retained (generally in dry form) in the conjugate zone prior to use, but will be free to migrate with the liquid sample (which leads to their reconstitution or activation). For example, in LFDs which are based on a porous material substrate, the test sample will be taken up in the sample receiving zone and then drawn through the porous material to the conjugate zone. When the porous material of the conjugate zone is moistened, the labelled first specific binding partners will be free to bind to the analyte (if present) and they are then transported to the detection zone.

Hence, in the conjugate zone, the first specific binding partners will bind to the analyte, if any analyte is present in the test sample. The liquid sample is then drawn by capillary action or active fluid flow to the next zone.

An appropriate transfer fluid (e.g. aqueous solution) may be used to transfer the first specific binding partners to the detection zone, e.g. from a sample receiving zone.

(c) A detection zone. This zone may comprise one or more second specific binding partners for the analyte. The second specific binding partners are immobilised, i.e. they cannot be mobilised by the action of the liquid test sample. Generally, the second specific binding partners are not linked to a detectable label. The second specific binding partners may comprise the same or different analyte-binding moieties as the first specific binding partner.

In some embodiments, the detection zone receives the analyte (sample) directly and the analyte is immobilised in the detection zone. In such embodiments, second specific binding partners are not used. The detection zone may comprise a size exclusion zone, as defined above. In such embodiments, the exosomes in the analyte (sample) pass through the size exclusion zone before being immobilised in the detection zone.

The first and second binding partners (when both are present) may participate in either a “sandwich” or a “competition” assay.

(d) Optionally, the lateral flow device may comprise a control zone, which provides a positive or negative control for the binding reaction. For example, the control zone may comprise immobilised trophoblast glycoprotein (TPGB).

(e) Optionally, the lateral flow device may comprise an absorbent zone. This acts as a sink for the liquid sample and/or transfer fluid.

The liquid sample is generally drawn through the device of the invention (e.g. a lateral flow device, vertical flow device or microfluidics device) by capillary action (or “wicking”) or is actively transported (e.g. using a pump) to the next zone.

In all embodiments of the invention, an appropriate transfer fluid (e.g. aqueous solution) may be used to transfer the moieties (e.g. the biological sample or the first or second specific binding partners) between the zones. The methods of the invention may therefore additionally comprise the step of applying a transfer fluid to any of the zones referred to herein.

In some embodiments, the sample is transported by active fluid flow (of a transfer fluid which may comprise the biological sample or exosomes) from the sample or liquid receiving zone to the subsequent zones, or from one zone to another.

For example, a pump (e.g. a mechanical pump) may be used to transport the transfer fluid which may comprise the biological sample or exosomes. Such a pump may be used to increase the flow rate of the transfer fluid and/or the speed of exosome isolation. The pump may be located at any suitable position within the device, e.g. at the end of the device (e.g. after the last zone or sink).

In one embodiment, the lateral flow device includes discrete zones (a)-(c), and optionally (d) and (e), which are in fluid communication with one another, in this order.

In this embodiment, the sample is applied to the sample receiving zone (a).

In another embodiment, the lateral flow device comprises zones (a), (b), (c) and optionally (d) and (e), which are in fluid communication with one another, in this order.

In this embodiment, the sample is applied directly to the detection zone (c) and an appropriate transfer fluid is applied to the sample receiving zone (a).

In this way, the biological sample or transfer fluid progresses from the sample receiving zone (or transfer fluid receiving zone), through the conjugate zone and into the detection zone, and optionally through the control zone and/or to the absorbent zone.

In one embodiment, the LFD may comprise a porous planar substrate or solid support comprising one or more discrete zones as defined herein.

In one simple form, the LFD (or the porous planar substrate or the solid support) comprises a porous strip or chromatographic strip comprising a one or more discrete zones (as defined herein), along which the liquid test sample may be drawn by capillary action or active transport.

The strip may, for example, be paper, nitrocellulose, polyvinylidene fluoride, nylon or polyethersulfone. The use of such strips is well known in the art.

In other embodiments, the LFD comprises a device having one or more flow paths or channels in fluid communication with and between one or more discrete zones (e.g. (a)-(e) as described above). The device may be a vertical flow device or microfluidic device. It may additionally comprise a pump, i.e. to move the fluids between the zones.

A typical LFD comprises a hollow casing constructed of moisture-impervious solid material (which may be opaque or transparent, but will generally include visually-readable portions at detection and control Zones) comprising a dry porous carrier which communicates directly or indirectly with the exterior of the casing such that a liquid test sample can be applied to the porous carrier at the sample receiving zone and be transported to the other zones.

Vertical flow devices often share one or more features in common with lateral flow devices, wherein the fluid transfer is generally by gravity (as opposed to capillary wicking). Alternatively, the vertical flow device may comprise a pump (e.g. at the end of the device) to aid flow of the transfer fluid. The vertical flow device may comprise one or more or all of the following zones:

(a) A sample receiving zone. This zone receives the test sample comprising the analyte (e.g. liquid sample) to be tested for.

The liquid sample (e.g. blood plasma or raw exosome sample) is then drawn by gravity to the next zone.

(b) Exosome isolation zone. This zone separates the exosomes from other cell vesicles, e.g. by size exclusion chromatography, e.g. through a 30-70 nm porous material, allowing exosomes to pass through.

(c) Detection zone. This zone may comprise one or more second specific binding partners for the analyte. The second specific binding partners are immobilised, i.e. they cannot be mobilised by the action of the liquid test sample. Generally, these second specific binding partners are not linked to a detectable label.

The presence of exosomes in the detection zone may be determined by the use of first specific binding partners. The first specific binding partners may be specific for the biomarkers or for exosomes. Alternatively, a general detection means may be used such a silver stain.

In yet a further embodiment, therefore, the invention provides a method of obtaining an indication of the presence of lung cancer in a subject, the method comprising the steps:
(a) immobilising a biological sample obtained from the subject in a detection zone on a solid support;
(b) contacting the solid support (preferably the detection zone) with one or more first specific binding partners, wherein each first specific binding partner specifically binds to a biomarker selected from the group consisting of PLD3, MAGE4A, GAGE2D, MTAP and UCHL1 polypeptides, and wherein each first specific binding partner is linked to a detectable label; and
(c) detecting the presence or absence of bound label in the detection zone, wherein the presence of bound label in the detection zone is indicative of the presence of lung cancer in the subject.

In the solid supports and lateral flow or vertical flow devices referred to herein, the detectable labels which are linked to the first specific binding partners may be the same or different.

In yet a further embodiment, therefore, the invention provides a method of obtaining an indication of the presence of lung cancer in a subject, the method comprising the steps:
(a) contacting a biological sample obtained from the subject with a solid support, wherein the solid support comprises a detection zone comprising one or more (e.g. 1, 2, 3, 4, or 5) immobilised specific binding partners against one or more biomarkers selected from the group consisting of PLD3, MAGE4A, GAGE2D, MTAP and UCHL1 polypeptides; and
(b) detecting the presence or absence of bound specific binding partners in the detection zone,

wherein the presence of bound specific binding partners in the detection zone is indicative of the presence of lung cancer in the subject.

For example, the solid support may comprise a sample receiving zone which is in fluid communication with the detection zone. The sample may travel from the sample receiving zone to the detection zone by active or passive fluid transfer, with or without use of a transfer fluid.

The specific binding partners are preferably antibodies which are independently specific for one of the biomarkers. The presence of bound specific binding partners in the detection zone may be detected by, for example, labelled secondary antibodies. (As used in this context, the term “bound specific binding partners” refers to specific binding partners (e.g antibodies) which are bound to an antigen, e.g. PLD3, MAGE4A, GAGE2D, MTAP or UCHL1 polypeptides.)

The invention also provides a solid support to which is immobilised one or more (e.g. 1, 2, 3, 4 or 5) first specific binding partners, wherein each first specific binding partner specifically binds to a biomarker selected from the group consisting of PLD3, MAGE4A, GAGE2D, MTAP and UCHL1 polypeptides. In some embodiments, the biomarker is selected from the group consisting of PLD3 and MTAP. Preferably, the biomarker is in the configuration in which it is presented on lung cancer exosomes.

In a further embodiment, the invention provides a method of obtaining an indication of the presence of lung cancer in a subject, the method comprising the steps:

(a) contacting a lateral flow device, vertical flow device or microfluidics device comprising a conjugate zone and a detection zone with a biological sample obtained from the subject, wherein
(i) the conjugate zone comprises one or more first specific binding partners, wherein each first specific binding partner specifically binds to a biomarker selected from the group consisting of PLD3, MAGE4A, GAGE2D, MTAP, and UCHL1 polypeptides, wherein each first specific binding partner is bound to a detectable label, and wherein the first specific binding partners are not immobilised in the conjugate zone; and
(ii) the biological sample is applied to the detection zone, and

(b) detecting the presence or absence of bound label in the detection zone, wherein the presence of bound label in the detection zone is indicative of the presence of lung cancer in the subject.

The biological sample is immobilised in the detection zone.

The detection zone and conjugate zone are in fluid communication.

The sample may travel from the conjugate zone to the detection zone by active or passive fluid transfer, with or without a transfer fluid.

Any of the devices disclosed herein may additionally comprise a size exclusion zone to enable isolation of exosomes and/or a pump to aid fluid transfer.

The invention also provides a lateral flow device, vertical flow device or microfluidics device comprising a conjugate zone and a detection zone, wherein
(i) the conjugate zone comprises one or more first specific binding partners, wherein each first specific binding partner specifically binds to a biomarker selected from the group consisting of PLD3, MAGE4A, GAGE2D, MTAP and UCHL1 polypeptides, wherein each first specific binding partner is bound to a detectable label, and wherein the first specific binding partners are not immobilised in the conjugate zone; and
(ii) the detection zone is adapted to receive a biological sample.

The conjugate zone and detection zone are in fluid communication.

Preferably, wherein the lateral flow device, vertical flow device or microfluidics comprises:
(a) a transfer fluid receiving zone, to which an aqueous solution is applied or is capable of being applied;
(b) a conjugate zone, as defined above;
(c) a detection zone, to which the biological sample is applied or is capable of being applied;
and optionally one or both of:
(d) a control zone, and
(e) an absorbent zone,

wherein the above zones, when present, are joined in (fluid) communication, in the above-mentioned order.

The device may also comprise a size exclusion zone prior between (a) and (b).

The lateral flow device, vertical flow device or microfluidics device may also comprise an exosome isolation zone which is joined (in fluid communication) with (prior to) the detection zone. In this embodiment, transfer fluid which is applied to the fluid receiving zone is carried into the conjugate zone; the first specific binding partners in the conjugate zone are carried into the detection zone where they will bind to the biological sample if any of the selected biomarkers are present in the detection zone. Optionally, the biological sample is first applied to the exosome isolation zone, where exosomes are isolated and then passed to the detection zone.

In yet another embodiment, the invention provides a method of obtaining an indication of the presence of lung cancer in a subject, the method comprising the steps:
(a) contacting a lateral flow device, vertical flow device or microfluidics device comprising a conjugate zone and a detection zone with a biological sample obtained from the subject, wherein
   (i) the conjugate zone comprises one or more first specific binding partners, wherein each first specific binding partner is bound to a detectable label,
   wherein the first specific binding partners are not immobilised in the conjugate zone; and
   (ii) the detection zone comprises one or more second specific binding partners, wherein each second specific binding partner is immobilised in the detection zone,

wherein the first specific binding partners and/or the second specific binding partners each specifically bind to a biomarker selected from the group consisting of PLD3, MAGE4A, GAGE2D, MTAP and UCHL1 polypeptides, and wherein first or second specific binding partners which do not specifically bind to one of said biomarkers bind to a ligand which is present in exosomes; and
(b) detecting the presence or absence of bound label in the detection zone, wherein the presence of bound label in the detection zone is indicative of the presence of lung cancer in the subject.

The conjugate zone and detection zone are in fluid communication.

The invention also provides a lateral flow device, vertical flow device or microfluidics device comprising a conjugate zone and a detection zone, wherein
(a) the conjugate zone comprises one or more first specific binding partners, wherein each first specific binding partner is linked to a detectable label, and wherein the first specific binding partners are not immobilised in the conjugate zone;
(b) the detection zone comprises one or more second specific binding partners, wherein the second specific binding partners are immobilised in the detection zone,
characterised in that the first specific binding partners and/or the second specific binding partners each specifically bind to a biomarker selected from the group consisting of PLD3, MAGE4A, GAGE2D, MTAP and UCHL1 polypeptides.

The conjugate zone and detection zone are in fluid communication.

Preferably, the lateral flow device, vertical flow device or microfluidics device comprises:
(a) a sample receiving zone, to which the biological sample is applied or is capable of being applied;
(b) a conjugate zone, wherein each first specific binding partner is linked to a detectable label, and wherein the first specific binding partners are not immobilised;
(c) a detection zone, wherein the second specific binding partners are immobilised in the detection zone;

and optionally one or both of:
(d) a control zone, and
(e) an absorbent zone,

wherein the above zones (when present) are joined in (fluid) communication, in the above-mentioned order.

The lateral flow device, vertical flow device or microfluidics device may also comprise an exosome isolation zone which is joined (in fluid communication) with (prior to) the sample receiving zone or between the sample receiving zone and the conjugate zone.

In this embodiment, the biological sample which is applied to the sample receiving zone is carried into the conjugate zone (e.g. by a transfer fluid). Optionally, the biological sample is first applied to the exosome isolation zone, where exosomes are isolated and are then passed to the sample receiving zone. One or more of the first specific binding partners may bind to the biomarkers in the biological sample in the conjugate zone to form a sample/first binding partner complex. These binding complexes are carried into the detection zone (e.g. by a transfer fluid). In the detection zone, these binding complexes may bind to immobilised second specific binding partners. In this way, detectable label is bound in the detection zone, where it may be detected.

The transfer fluid may comprise the biological sample. The biological sample will be in liquid form, preferable an aqueous liquid. The transfer fluid and/or biological sample may additionally comprise a pharmaceutically-acceptable diluent, carrier or excipient. The transfer fluid and/or biological sample may also comprise suitable amounts and concentrations of buffers, salts, surfactants and/or blocking agents. These may be used to enhance the sensitivity and/or specificity of the methods of the invention.

The analyte to be tested for (i.e. the biological sample) may be one which comprises or is suspected of comprising one or more of PLD3, MAGE4A, GAGE2D, MTAP and UCHL1 polypeptides.

In practical terms, the biological sample will be obtained from the subject, and the exosomes and/or polypeptides obtained therefrom may then be tested (as the analyte), optionally in isolated form or isolated by a device of the invention.

Each first specific binding partner is preferably a polypeptide-binding moiety, linked to a detectable label.

The polypeptide-binding moiety may be a specific or a non-specific polypeptide binding moiety.

In some embodiments, each first specific binding partner is an antibody or antigen-binding portion thereof which specifically binds to one of the biomarkers defined herein.

In some embodiments, each second specific binding partner is an antibody or antigen-binding portion thereof which specifically binds to one of the biomarkers defined herein.

In some embodiments, the first or second specific binding partners which do not specifically bind to one of said biomarkers may bind to a ligand which is commonly present in exosomes or to polypeptides in general. In such embodiments, it is preferable that the first specific binding partners specifically bind to one of said biomarkers.

The label facilitates the detection of the analyte (sample) if the first or second specific binding partner/analyte complex is bound in the detection zone. The label may, for example, be selected from the group consisting of fluorescence tags, dye labels, enzyme reporters, biotin, epitope tags, metal nanoparticles, carbon, coloured latex nanoparticles, magnetic beads, fluorescence beads, and coloured polystyrene beads.

In some embodiments, the detectable label is a region of the first or second specific binding partner, e.g. an antibody Fc domain, which is detectable through the use of a secondary antibody. Preferably, the label is an optically-detectable marker (i.e. detectable by eye). In some embodiments, the label is a magnetic bead.

In some embodiments, the label has a known density value; this may facilitate the quantification of the marker in the detection zone.

The detectable label may be a multivalent scaffold. As used herein, the term “multivalent scaffold” refers to a support to which a plurality of linkers, as disclosed herein, may be chemically attached or anchored. Examples of multivalent scaffolds include nanoparticles, hyper-branched polymers and cyclodextrins.

Preferably, the method steps are carried out in the order specified.

The disclosure of each reference set forth herein is specifically incorporated herein by reference in its entirety.

### BRIEF DESCRIPTION OF THE FIGURES

FIG. 1. Examples of lateral flow (FIG. 1A) and vertical flow (FIG. 11B) detection devices for lung cancer specific surface exosomal antigens.

FIG. 2.

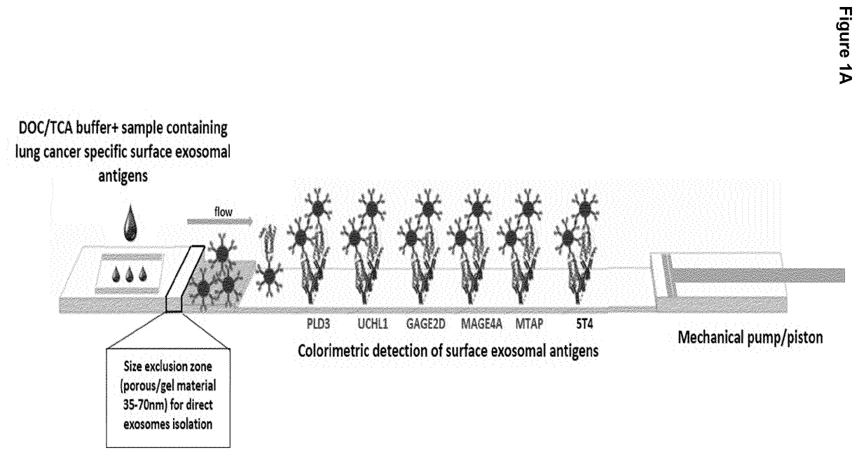

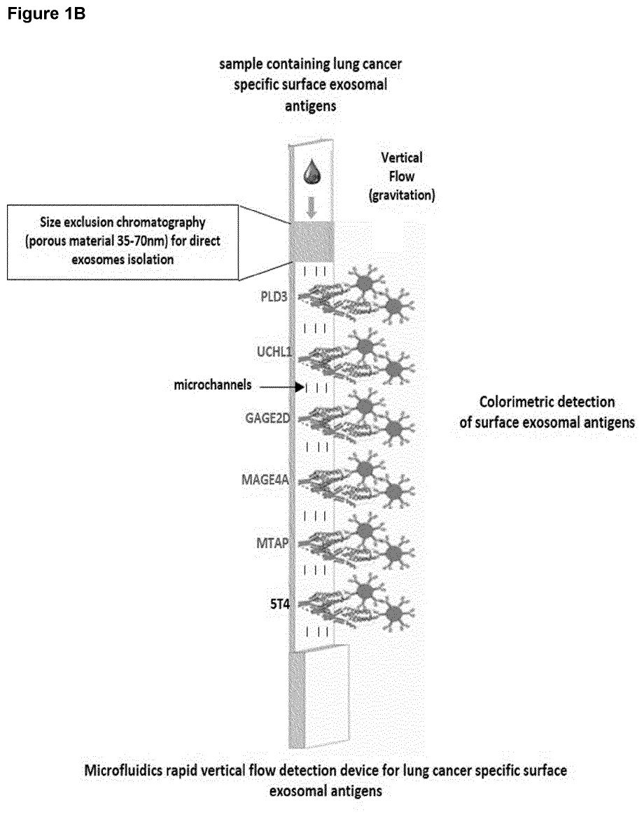

A. Nanoparticle Tracking Analysis (NTA) of H1299 cells displaying the size distribution of exosomes isolated from conditional media.

B. Western blot analysis of exosomal surface proteins. After exosomes were treated with trypsin to cleave surface proteins, the supernatant was collected and exposed by DOC/TCA to precipitate cleaved proteins.

C. Immunoprecipitation (IP) of exosomal surface proteins and analysis by Western blot technique.

D. ELISA analysis of lung cancer exosomes. Concentration of exosomal surface proteins, after exosomes were isolated from plasma in lung cancer patients in advanced stages (IV and III) with metastatic dissemination, labelled as “mets.”, and in early stages (I, II) without metastatic dissemination, labelled as “early”.

E. ELISA analysis of lung cancer exosomes for exosomal markers CD9 and CD81.

Fibronectin was added as a positive control.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The following Materials and Methods were used in one or more of the following Examples.

Material and Methods

Isolation of Exosomes

H1299 cells (1×10⁷ cells/condition) were grown for the indicated times, up to 3 days, in DMEM supplemented with 0.1% (v/v) FBS (depleted of bovine exosomes and extracellular vesicles by overnight centrifugation at 100,000×g), 2 mM Glutamine and 100 U/ml Pen/Strep. Conditioned medium was collected and EVs were isolated by sequential ultracentrifugation at 2000×g for 40 min, 10,000×g for 60 min, 100,000×g for 1.5 h in an Optima XPN-80 (Beckman Coulter) ultracentrifuge using UltraClear Thinwall tubes. The exosomes were washed once in 1 ml of PBS and purified by centrifugation at 100,000×g for 80 min in an Optima MAX-XP Ultracentrifuge (Beckman Coulter).

Plasma from clinical samples from lung cancer patients were centrifuged at 2500×g for 15 minutes, followed by filtration through 0.8 μm filter and centrifuged at 10000×g for 40 minutes. Precleared EVs were filtered through 0.22 μm filter and centrifuged at 100,000×g for 1.5 h in an Optima XPN-80 (Beckman Coulter) ultracentrifuge using UltraClear Thinwall tubes. Exosomes were further isolated by size exclusion chromatography, by using IZON columns (with pores 35 nm) and again centrifuged at 100.000×g for 80 minutes in an Optima XPN-80 (Beckman Coulter) ultracentrifuge using UltraClear Thinwall tubes. Protein levels from both type of exosomes (from cell culture and from clinical samples were measured using MicroBCA assay (Thermo Scientific and used for further analyses).

Nanoparticle Tracking Analysis (NTA)

Total exosomal pellet was), vigorously resuspended by pipetting in 1 mL PBS (Gibco and kept on ice, before starting the analysis.

Before starting any measurement, NanoSight NS300 (Malvern Panalytical) was washed three times by loading distilled water onto a syringe pump using a 1 mL syringe and pressing the liquid into the flow-cell top plate of the NanoSight. PBS was used to prime the instrument and to control the purity of the diluent (i.e. absence of particulate in the solution or presence of particulate in a concentration lower than detectable level). After priming, 1 mL of sample was carefully loaded on the syringe pump. Every measurement was done automatically, with the aid of the syringe pump and a script for data acquisition was generated on the NTA 3.2 software. Three recordings of 60 min each were automatically taken once each sample was loaded in the chamber and the focus on the particles in solution was adjusted manually.

Sample Preparation for WBs and IP

H1299 exosomes resuspended in PBS and treated or not with Trypsin for 30 minutes in 37 C. After treatment, the exosomes were ultra-centrifuged at 100,000×g for 80 minutes to separate exosomes from cleaved surface proteins. Collected supernatant (that contained cleaved surface exosomal proteins) was exposed by treatment with sodium deoxycholate/trichloroacetic acid (DOC/TCA) to precipitate proteins. (Briefly, samples were incubated with 2% of Na-deoxycholate for 15 minutes at room temperature, followed by treatment with 24% trichloracetic acid. Samples were centrifuged, pellet washed twice with cold acetone and resuspended in 1×PBS.) Exosomes and precipitated supernatant were resuspended in PBS for either Western blot analyses or immunoprecipitation with specific antibodies against certain surface proteins.

SDS-PAGE and Western Blot

Exosomes and precipitated supernatant were resuspended in PBS. The samples were then boiled at 100° C. for 10 min. Samples were normalized with 1× Loading buffer (Thermo Scientific), so that equal loading per experiment was achieved. Protein samples were loaded onto a gel and separated by SDS-PAGE using NuPAGE® pre-cast gels (10% or 4-12%) (Thermo Scientific). Protein was transferred onto PVDF membrane and blocked and incubated with primary antibody in 3% non-fat milk diluted in PBS-Tween 20. Secondary antibodies were incubated in 3% non-fat milk.

Membranes were covered in ECL solutions from Thermo Scientific, Millipore or GE Healthcare prior to exposure to film (Fujifilm) and developed in a XoGraph developer.

Indirect ELISA

Isolated exosomes (1 μg/ml) were diluted in carbonate buffer, pH 9.4 and coated on microtiter plate overnight at 4C. After extensive washing and blocking for 1h at room temperature, exosomes were incubated with primary antibody (1 μg/ml) for 2 hours at room temperature. After washing, exosomes were incubated with secondary antibody for 1h at room temperature, followed by washing and TMB substrate incubation for 30 minutes at room temperature in the dark. The HRP reaction was stop by adding stop solution and OD was read on microplate reader at 450 nm. The concentration of proteins in exosomes from human lung cancer plasma was calculated by Graphpad software. Statistic was calculated by Student's t-test with significant p-values 50.05.

### Example 1: Identification of Exosomal Biomarkers for Lung Cancer

We identified 22 proteins by LC-MS/MS exosomal protein profiling which were specific for a lung cancer cell line (H1299) and that were not detected in other types of cancer cell lines.

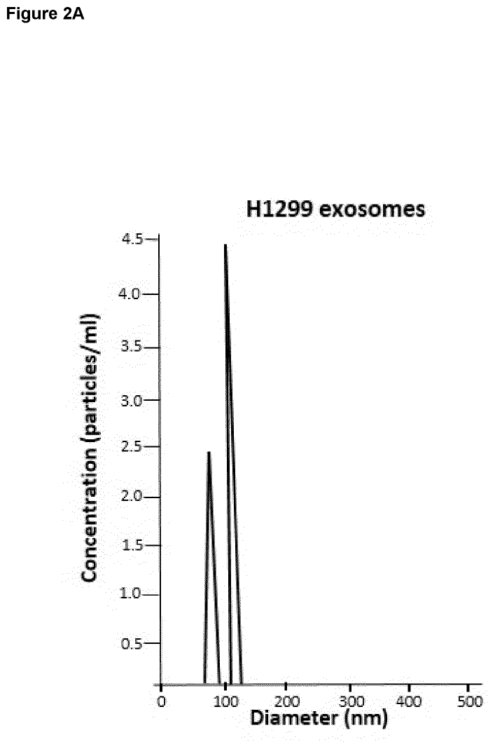

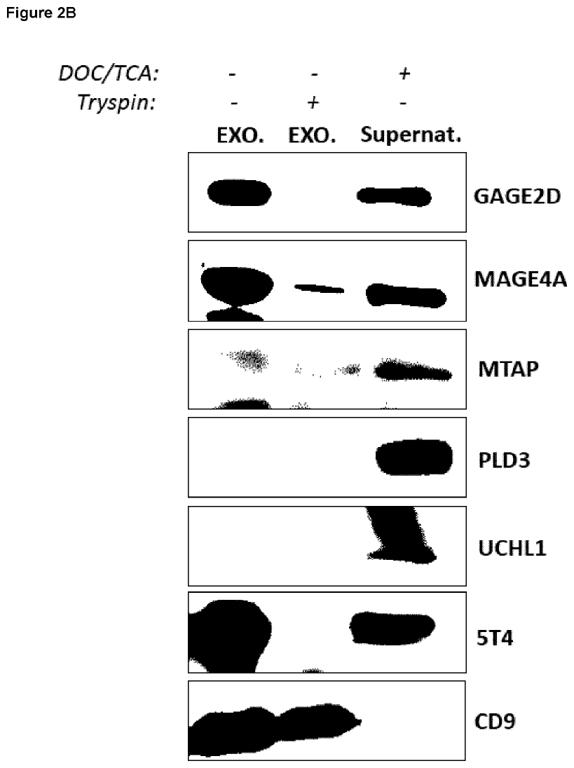

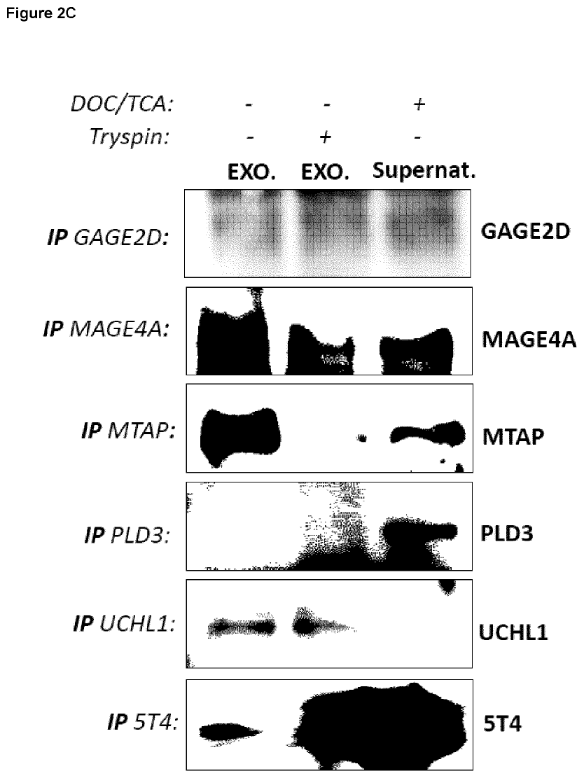

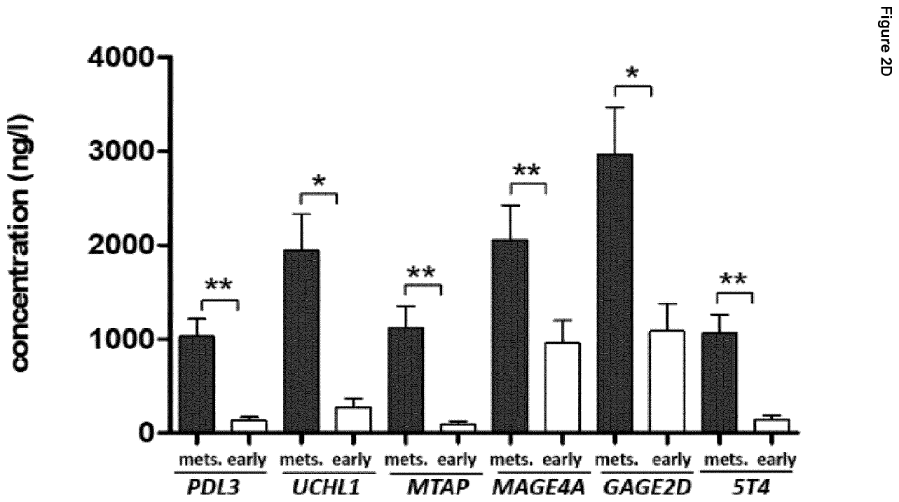

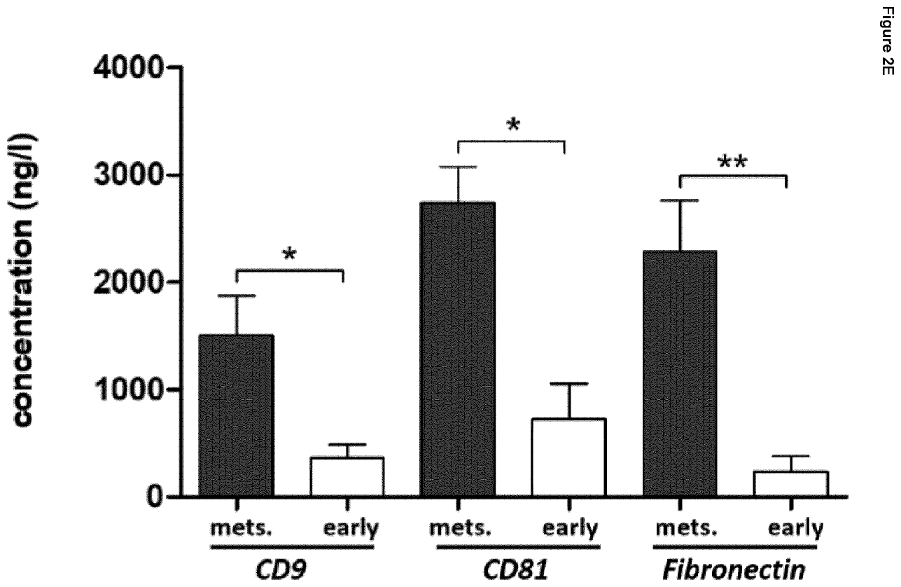

To characterize whether our identified proteins were localized on the surface of lung cancer exosomes, isolated exosomes were verified by NTA analyses to confirm their size (FIG. 2A) and followed by trypsin treatment to cleave proteins from their surface.

In order to increase the concentration of exosomal surface proteins, the proteins in the supernatant after trypsin treatment were precipitated by DOC/TCA and were verified by Western blot. Five lung cancer specific proteins were identified in the precipitated samples (FIG. 2B): MAGE4A, GAGE2D, MTAP, PLD3 and UCHL1. After trypsinisation, the exosomes had highly decreased expression of all of these proteins, confirming that these proteins were proteolytically-cleaved (liberated) from the exosomal surface.

Interestingly, Western blot analysis did not detect any expression of UCHL1 and PLD3 proteins in control exosomes (PBS), suggesting that their concentration was below the detection limit of this method (FIG. 2B).

These results clearly showed that the identified proteins are localized on the surfaces of exosomes and that these markers may be used for quick diagnostic tests for the precise and accurate detection of even low concentration of proteins in the lung cancer samples.

### Example 2: Immunoprecipitation of Biomarkers

To further confirm our results, we again treated the exosomes with or without trypsin, followed by immunoprecipitation of the cleaved proteins with antibodies which were specific to the identified surface proteins. The control samples and supernatants after enzymatic treatment and precipitated with DOC/TCA solution showed expression of all surface markers compared to treated exosomes by Trypsin (FIG. 2c). Interestingly, PLD3 expression was again detectable only in DOC/TCA-precipitated supernatants, suggesting DOC/TCA solution is important for increasing concentration of some low abundance proteins.

These results clearly showed that the identified proteins are localized on the surfaces of exosomes. These proteins may therefore be used as markers in quick diagnostic tests for the precise and accurate detection of even low concentrations of proteins in the lung cancer samples.

### Example 3: Determination of Exosomal Lung Cancer Specific Biomarkers in Exosomes from Lung Cancer Patients in Early (I-11) and Advanced, Metastatic (III-IV) Stages

To investigate whether the specific lung cancer biomarkers identified in vitro (FIG. 2B, 2C) were present in exosomes from liquid biopsies of lung cancer patients, we collected 5 ml of blood plasma from lung cancer patients in stages I-IV and isolated exosomes. The results clearly showed not only presence of lung cancer specific biomarkers in exosomes from lung cancer patients, but also significant differences between early stages and advanced stages with metastatic dissemination (FIG. 2D). Different concentrations of the identified lung cancer specific proteins confirm their role as a potential exosomal biomarkers which are capable of distinguishing early stages of lung cancer in the patients without metastasis from patients with developed lung cancer metastatic disease. The exosomes isolated from liquid biopsies were confirmed by general exosomal markers CD9, CD81 and for Fibronectin as a positive controls (FIG. 2E).

### REFERENCES

Bebelman, M. P., Smit, M. J., Pegtel, D. M., Baglio, S. R., 2018. Biogenesis and function of extracellular vesicles in cancer. Pharmacol. Ther. 188, 1-11. https://doi.org/10.1016/j.pharmthera.2018.02.013
Cazzoli, R., Buttitta, F., Di Nicola, M., Malatesta, S., Marchetti, A., Pass, H. I., 2013. MicroRNAs derived from circulating exosomes as non-invasive biomarkers for screening and diagnose lung cancer. J. Thorac. Oncol. Off. Publ. Int. Assoc. Study Lung Cancer 8, 1156-1162. https://doi.org/10.1097/JTO.0b013e318299ac32
Cristiano, S., Leal, A., Phallen, J., Fiksel, J., Adleff, V., Bruhm, D. C., Jensen, S. Ø., Medina, J. E., Hruban, C., White, J. R., Palsgrove, D. N., Niknafs, N., Anagnostou, V., Forde, P., Naidoo, J., Marrone, K., Brahmer, J., Woodward, B. D., Husain, H., van Rooijen, K. L., Ørntoft, M.-B. W., Madsen, A. H., van de Velde, C. J. H., Verheij, M., Cats, A., Punt, C. J. A., Vink, G. R., van Grieken, N. C. T., Koopman, M., Fijneman, R. J. A., Johansen, J. S., Nielsen, H. J., Meijer, G. A., Andersen, C. L., Scharpf, R. B., Velculescu, V. E., 2019. Genome-wide cell-free DNA fragmentation in patients with cancer. Nature 570, 385-389. https://doi.org/10.1038/s41586-019-1272-6
Devarakonda, S., Morgensztern, D., Govindan, R., 2015. Genomic alterations in lung adenocarcinoma. Lancet Oncol. 16, e342-e351. https://doi.org/10.1016/S1470-2045(15)00077-7
Diehl, F., Schmidt, K., Choti, M. A., Romans, K., Goodman, S., Li, M., Thornton, K., Agrawal, N., Sokoll, L., Szabo, S. A., Kinzler, K. W., Vogelstein, B., Diaz, L. A., 2008. Circulating mutant DNA to assess tumor dynamics. Nat. Med. 14, 985-990. https://doi.org/10.1038/nm.1789
Fan, H., Shao, Z.-Y., Xiao, Y.-Y., Xie, Z.-H., Chen, W., Xie, H., Qin, G.-Y., Zhao, N.-Q., 2015. Incidence and survival of non-small cell lung cancer in Shanghai: a population-based cohort study. BMJ Open 5, e009419. https://doi.org/10.1136/bmjopen-2015-009419
Fan, T. W. M., Zhang, X., Wang, C., Yang, Y., Kang, W.-Y., Arnold, S., Higashi, R. M., Liu, J., Lane, A. N., 2018. Exosomal lipids for classifying early and late stage non-small cell lung cancer. Anal. Chim. Acta 1037, 256-264. https://doi.org/10.1016/j.aca.2018.02.051
Jakobsen, K. R., Paulsen, B. S., Bæk, R., Varming, K., Sorensen, B. S., Jorgensen, M. M., 2015. Exosomal proteins as potential diagnostic markers in advanced non-small cell lung carcinoma. J. Extracell. Vesicles 4. https://doi.org/10.3402/jev.v4.26659 Johnstone, R. M., Adam, M., Hammond, J. R., Orr, L., Turbide, C., 1987. Vesicle formation during reticulocyte maturation. Association of plasma membrane activities with released vesicles (exosomes). J. Biol. Chem. 262, 9412-9420.
Kumarakulasinghe, N. B., Zanwijk, N. van, Soo, R. A., 2015. Molecular targeted therapy in the treatment of advanced stage non-small cell lung cancer (NSCLC). Respirology 20, 370-378. https://doi.org/10.1111/resp.12490
Lee, S., Choi, E. K., Chung, W. K., Shin, K. H., Ahn, S. D., Kim, J. H., Kim, S.-W., Suh, C., Lee, J. S., Kim, W. S., Kim, D. S., Kim, D. K., Park, S. I., Sohn, K.-H., 2002. Postoperative adjuvant chemotherapy and radiotherapy for stage II and III non-small cell lung cancer (NSCLC). Lung Cancer 37, 65-71. https://doi.org/10.1016/S0169-5002(02)00026-0
Levy, B., Hu, Z. I., Cordova, K. N., Close, S., Lee, K., Becker, D., 2016. Clinical Utility of Liquid Diagnostic Platforms in Non-Small Cell Lung Cancer. The Oncologist 21, 1121-1130. https://doi.org/10.1634/theoncologist.2016-0082
Li, Y., Zhang, Y., Qiu, F., Qiu, Z., 2011. Proteomic identification of exosomal LRG1: A potential urinary biomarker for detecting NSCLC. ELECTROPHORESIS 32, 1976-1983. https://doi.org/10.1002/elps.201000598
National Lung Screening Trial Research Team, Aberle, D. R., Adams, A. M., Berg, C. D., Black, W. C., Clapp, J. D., Fagerstrom, R. M., Gareen, I. F., Gatsonis, C., Marcus, P. M., Sicks, J. D., 2011. Reduced lung-cancer mortality with low-dose computed tomographic screening. N. Engl. J. Med. 365, 395-409. https://doi.org/10.1056/NEJMoa1102873 Newman, A. M., Bratman, S. V., To, J., Wynne, J. F., Eclov, N. C. W., Modlin, L. A., Liu, C. L., Neal, J. W., Wakelee, H. A., Merritt, R. E., Shrager, J. B., Loo, B. W., Alizadeh, A. A., Diehn, M., 2014. An ultrasensitive method for quantitating circulating tumor DNA with broad patient coverage. Nat. Med. 20, 548-554. https://doi.org/10.1038/nm.3519
Siegel, R. L., Miller, K. D., Jemal, A., 2017. Cancer statistics, 2017. CA. Cancer J. Clin. 67, 7-30. https://doi.org/10.3322/caac.21387
Song, Z., Wang, S., Liu, Y., 2018. The diagnostic accuracy of liquid exosomes for lung cancer detection: a meta-analysis. OncoTargets Ther. 12, 181-192. https://doi.org/10.2147/OTT.S188832
Sourvinou, I. S., Markou, A., Lianidou, E. S., 2013. Quantification of circulating miRNAs in plasma: effect of preanalytical and analytical parameters on their isolation and stability. J. Mol. Diagn. JMD 15, 827-834. https://doi.org/10.1016/j.jmoldx.2013.07.005
Sun, Y., Huo, C., Qiao, Z., Shang, Z., Uzzaman, A., Liu, S., Jiang, X., Fan, L.-Y., Ji, L., Guan, X., Cao, C.-X., Xiao, H., 2018. Comparative Proteomic Analysis of Exosomes and Microvesicles in Human Saliva for Lung Cancer. J. Proteome Res. 17, 1101-1107. https://doi.org/10.1021/acs.jproteome.7b00770
van der Pol, E., Boing, A. N., Harrison, P., Sturk, A., Nieuwland, R., 2012. Classification, functions, and clinical relevance of extracellular vesicles. Pharmacol. Rev. 64, 676-705. https://doi.org/10.1124/pr.112.005983
Vanni, I., Alama, A., Grossi, F., Dal Bello, M. G., Coco, S., 2017. Exosomes: a new horizon in lung cancer. Drug Discov. Today 22, 927-936. https://doi.org/10.1016/j.drudis.2017.03.004
Wong, S. Q., Li, J., Tan, A. Y.-C., Vedururu, R., Pang, J.-M. B., Do, H., Ellul, J., Doig, K., Bell, A., MacArthur, G. A., Fox, S. B., Thomas, D. M., Fellowes, A., Parisot, J. P., Dobrovic, A., 2014. Sequence artefacts in a prospective series of formalin-fixed tumours tested for mutations in hotspot regions by massively parallel sequencing. BMC Med. Genomics 7, 23. https://doi.org/10.1186/1755-8794-7-23
Yamashita, T., Kamada, H., Kanasaki, S., Maeda, Y., Nagano, K., Abe, Y., Inoue, M., Yoshioka, Y., Tsutsumi, Y., Katayama, S., Inoue, M., Tsunoda, S., 2013. Epidermal growth factor receptor localized to exosome membranes as a possible biomarker for lung cancer diagnosis. Pharm. 68, 969-973.
Zhang, R., Xia, Y., Wang, Z., Zheng, J., Chen, Y., Li, X., Wang, Y., Ming, H., 2017. Serum long non coding RNA MALAT-1 protected by exosomes is up-regulated and promotes cell proliferation and migration in non-small cell lung cancer. Biochem. Biophys. Res. Commun. 490, 406-414. https://doi.org/10.1016/j.bbrc.2017.06.055

1-27. (canceled)

28. A method of treating lung cancer in a subject, the method comprising the steps of:
(a) obtaining an indication of the presence of lung cancer in a subject by a method comprising the step:
determining the presence of or level of one or more biomarkers selected from the group consisting of PLD3, MAGE4A, GAGE2D, MTAP and UCHL1 polypeptides within a biological sample obtained from the subject, wherein the biological sample comprises exosomes and/or polypeptides obtained therefrom,
wherein the presence of or level of one or more of the biomarkers within the biological sample is indicative of the presence of lung cancer in the subject;
and
(b) administering a treatment appropriate for treating lung cancer to the subject if an indication of the presence of lung cancer in the subject is obtained, thereby treating lung cancer in the subject.

29. The method as claimed in claim 28, the method comprising the steps:
(a) obtaining an indication of the presence of early stage lung cancer in a subject by a method comprising the step:
determining the presence of or level of one or more biomarkers selected from the group consisting of PLD3, MTAP and/or UCHL1 polypeptides within a biological sample obtained from the subject, wherein the biological sample comprises exosomes and/or polypeptides obtained therefrom, and
wherein the presence of PLD3, MTAP and/or UCHL1 polypeptides within the biological sample is indicative of the presence of early stage lung cancer (preferably stages I, II, or III) in the subject;
and
(b) administering a treatment appropriate for treating early stage lung cancer to the subject if an indication of the presence of early stage lung cancer in the subject is obtained, thereby treating early stage lung cancer in the subject.

30. The method as claimed in claim 28, the method comprising the steps:
(a) obtaining an indication of the presence of metastatic lung disease in a subject by a method comprising the step:
determining the presence of or level of one or more biomarkers selected from the group consisting of MAGE4A and GAGE2D polypeptides within a biological sample obtained from the subject, wherein the biological sample comprises exosomes and/or polypeptides obtained therefrom, and
wherein the presence of MAGE4A and/or GAGE2D polypeptides within the biological sample is indicative of the presence of metastatic lung disease in the subject;
and
(b) administering a treatment appropriate for treating metastatic lung disease to the subject if an indication of the presence of metastatic lung disease in the subject is obtained, thereby treating metastatic lung disease in the subject.

31. The method as claimed in claim 28, wherein the group of biomarkers consists of 1, 2, 3, 4, or 5 of PLD3, MAGE4A, GAGE2D, MTAP and UCHL1 polypeptides.

32. The method as claimed in claim 28, wherein the group of biomarkers consists of:
(i) PLD3;
(ii) MTAP; or
(iii) PLD3 and MTAP.

33. The method as claimed in claim 28, wherein the biological sample is a bodily fluid or a liquid biopsy from the subject.

34. The method as claimed in claim 28, wherein the biological sample is a sample of the subject's blood, saliva, bronchial lavage or urine, preferably a sample of subject's blood serum or blood plasma.

35. The method as claimed in claim 28, wherein the biological sample is a sample of:
(i) membrane-associates polypeptides which have been obtained from exosomes from the subject, preferably exosomes from the subject's blood; or
(ii) polypeptides which have been cleaved or released from the surface of exosomes from the subject, preferably exosomes from the subject's blood.

36. A method of treating lung cancer in a subject, the method comprising the steps of:
(a) obtaining an indication of the presence of lung cancer in a subject by a method comprising the steps:
(i) contacting a biological sample obtained from the subject with a solid support,
wherein the biological sample is a sample of isolated and/or purified exosomes which have been obtained from the subject, wherein the solid support comprises a detection zone comprising one or more (e.g. 1, 2, 3, 4, or 5) immobilised specific binding partners against one or more biomarkers selected from the group consisting of PLD3, MAGE4A, GAGE2D, MTAP and UCHL1 polypeptides; and
(ii) detecting the presence or absence of bound specific binding partners in the detection zone,
wherein the presence of bound specific binding partners in the detection zone is indicative of the presence of lung cancer in the subject;
and
(b) administering a treatment appropriate for treating lung cancer to the subject if an indication of the presence of lung cancer in the subject is obtained, thereby treating lung cancer in the subject.

37. The method as claimed in claim 36, wherein the group of biomarkers consists of 1, 2, 3, 4, or 5 of PLD3, MAGE4A, GAGE2D, MTAP and UCHL1 polypeptides.

38. The method as claimed in claim 36, wherein the group of biomarkers consists of:
(i) PLD3;
(ii) MTAP; or
(iii) PLD3 and MTAP.

39. The method as claimed in claim 36, wherein the biological sample is a bodily fluid or a liquid biopsy from the subject.

40. The method as claimed in claim 36, wherein the biological sample is a sample of the subject's blood, saliva, bronchial lavage or urine, preferably a sample of subject's blood serum or blood plasma.

41. The method as claimed in claim 36, wherein the biological sample is a sample of:
(i) membrane-associates polypeptides which have been obtained from exosomes from the subject, preferably exosomes from the subject's blood; or
(ii) polypeptides which have been cleaved or released from the surface of exosomes from the subject, preferably exosomes from the subject's blood.

42. A lateral flow device, vertical flow device or microfluidics device comprising a conjugate zone and a detection zone, wherein:
(a) the conjugate zone comprises one or more first specific binding partners,
wherein each first specific binding partner is linked to a detectable label, and
wherein the first specific binding partners are not immobilised in the conjugate zone; and
(b) the detection zone comprises one or more second specific binding partners,
wherein the second specific binding partners are immobilised in the detection zone, characterised in that the first specific binding partners and/or the second specific binding partners each specifically bind to a biomarker selected from the group consisting of PLD3, MAGE4A, GAGE2D, MTAP and UCHL1 polypeptides.

43. The device as claimed in claim 42, wherein the group of biomarkers consists of 1, 2, 3, 4, or 5 of PLD3, MAGE4A, GAGE2D, MTAP and UCHL1 polypeptides.

44. The device as claimed in claim 42, wherein the group of biomarkers consists of:
(i) PLD3;
(ii) MTAP; or
(iii) PLD3 and MTAP.

45. The device as claimed claim 42, wherein the lateral flow device additionally comprises a size exclusion zone which enables exosomes to be isolated from a biological sample based on their size.

46. The device as claimed in claim 45, wherein the biological sample is a bodily fluid or a liquid biopsy from the subject.

47. The device as claimed in claim 45, wherein the biological sample is a sample of the subject's blood, saliva, bronchial lavage or urine, preferably a sample of subject's blood serum or blood plasma.

48. The device as claimed in claim 45, wherein the biological sample is a sample of:
(i) membrane-associates polypeptides which have been obtained from exosomes from the subject, preferably exosomes from the subject's blood; or
(ii) polypeptides which have been cleaved or released from the surface of exosomes from the subject, preferably exosomes from the subject's blood.
